(19) United States
(12) Patent Application Publication
(10) Pub. No.: US 2005/0064051 A1
(43) Pub. Date: Mar. 24, 2005

(54) Oral Rinse

(76) Inventor: Laurier Falldien, Sudbury (CA)

Correspondence Address: D.W. EGGINS, 18 DOWNSVIEW DRIVE, BARRIE, ON, L4M 4P8, CA

(21) Appl. No.: 10/666,039

(22) Filed: Sep. 22, 2003

(51) Int. Cl. A61K 35/78

(52) U.S. Cl. 424/747; 424/769

(57) ABSTRACT

An oral treatment to promote prophylaxis and natural healing of inflammation sites by treatment of sites within the mouth, including pierce sites, sites of abrasion, periodontal and herpes inflammation sites, aptous ulcers, gingival inflammation, and “denture sore mouth” by the application of the given topical composition consisting of a soothing formulation containing Water; Vegetable Glycerine; Sea Salt; Peppermint Oil; and Tea Tree Oil. Application may be by mouth wash, by spray or pump, or by applicator.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO MICROFICHE APPENDIX

Not Applicable

### BACKGROUND OF THE INVENTION

1. This invention is directed to an oral rinse to promote prophylaxis, prevention of oral disease, and natural healing of inflammation sites, and to a method of treating inflammatory disorders of the mouth.

2. In the treatment of tongue-piercing inflammation sites within the mouth, use has been made of a soothing formulation containing Water; Vegetable Glycerine; Sea Salt; Peppermint Oil; and Tea Tree Oil.

### BRIEF SUMMARY OF THE INVENTION

A new use has been found for a known formulation, previously used in one embodiment as a soothing dressing when applied to a site of tongue piercing.

It has been found that this formulation is beneficial in promoting: healing in periodontal infection; oral bone regeneration; treatment of apthous ulcers, herpes simplex and gingivitis.

Further investigation is proceeding in the use of this formulation in the treatment of herpes, as manifested in mouth sores. Partially completed tests (which are in course of completion), have yielded positive results, which have been submitted for evaluation to a university Periodontal Department. That Department is including in its study the treatment of oral mucocytis using the same formulation.

A second, independent study, involves the treatment of Herpes Simplex, again using the same formulation.

The formulation of a batch consisted of:

| | | Weight % |
|---|---|---|
| water- | 66,192 mls; | 74.7 |
| vegetable glycerine- | 6720 mls; | 9.8 |
| sea salt- | 5040 mls; | 12.3 |
| peppermint oil- | 2.5 mls; (Mentha arvensis) | 1.7 |
| tea tree oil- | 2.5 mls. (Melaleuca alternifolia | 1.7 |

The invention further comprises a method of treating inflammatory disorders of the mouth, including pierce sites, sites of abrasion, periodontal and herpes inflammation sites, aptous ulcers, gingival inflammation, and “denture sore mouth” by the application of the given topical composition.

### DETAILED DESCRIPTION OF THE INVENTION

The following characteristics are attributed to the respective components of the formulation:
Water—serves as carrier and solvent;
Vegetable glycerine serves as solvent/dispersant and taste element;
Sea salt—antiseptic;
Peppermint oil—analgesic, antibiotic, antiseptic, anti-inflamatory;
Tea Tree oil—antiseptic, anti-fungal, anti-bacterial, anti-viral, immune system stimulant.

The formulation is prepared by adding the sea salt and vegetable glycerine to the water, in a stainless steel vessel, and stirring with a stainless steel paddle until the sea salt is dissolved. After passing the water/glycerine/sea salt solution through an ultra violet light field, the solution is then dispensed into amber coloured glass bottles, to minimize the adverse effects of light, to which tea tree oil is susceptible

The peppermint oil and tea tree oil are measured into a separate container, and there mixed. The mixed peppermint/tea tree oils are then accurately dispensed as a metered quantity into each of the respective glass bottles.

The bottles are then sealed and capped with a black phenolic cap having a low density polyethylene cone liner, and safety sealed, and then boxed.

The bottles include a direction to the user to thoroughly shake the bottle before use, in order to effectively mix the dispensed oils with the other constituents.

The formulation has been applied:
in the form of a mouthwash; or
by way of a spray; or by
direct application to affected areas, being painted on by way of a Q-Tip.
   Tests and Test Results

Herpes—herpal infections ranging from symptomatic onset, to sores ranging in diameter from 2-mm to 3-cm were treated with application of the topical formulation 3-4 times daily. Effectively 100% success rate of cure after 2-3 days treatment.

The symptomatic herpes onset is evidenced by tingling at the site, with no developed eruption. Termination of this tingling after due treatment was taken as evidence of a cure.

Aptous ulcers. Treatment as for Herpes (above), with corresponding rate of total success.

Gingival Proliferation (Denture Sore Mouth) due to ill-fitting dentures—characterized by the proliferation of oral mucosa. Treatment by topical application 3-times daily. This led to a 100% cure effected over 4-5 days of treatment. No surgery required.

Periodontitis—the presence of periodontal pockets in excess of 3 mm (3-mm pockets are considered normal). Treatment with topical application 3-4 times daily; led to 50% bone regeneration:
7 mm periodontal pockets diminished to 5 mm after treatment series;
5 mm periodontal pockets diminished to 4 mm after treatment series;
4 mm periodontal pockets diminished to 3 mm after treatment series;
   Microbial Tests

1. The subject formulation, identified herein as MDM, was applied by way of a spray. Microbial strain—Escherichia coli ATCC 25922. Lapsed time after treatment—1-hour.

Bacterial Enumeration (CFU per mililitre)

| | Colony 1 | colony 2 | colony 3 | Average | 1-hrLog reduct'n | Reduction % |
|---|---|---|---|---|---|---|
| | 5,100,000 | 6,000,000 | 4,800,000 | 5,3000,000 | 6.7 | 99.99 |
| Control | 0 | 0 | 0 | 1 | | |

2. The subject formulation, identified herein as MDM, was applied by way of a spray. Microbial strain—Staphylococcus aureus ATCC 25923. Lapsed time—1-hour.

Bacterial Enumeration (CFU per mililitre)

| | Colony 1 | colony 2 | colony 3 | Average | 1-hrLog reduct'n | Reduction % |
|---|---|---|---|---|---|---|
| | 4,900,000 | 4,700,000 | 4,300,000 | 4,633,333 | 6.7 | 99.99 |
| Control | 0 | 0 | 0 | 1 | | |

3. The subject formulation, identified herein as MDM, was applied by way of a spray. Microbial strain—Pseudomonas aeruginosa ATCC27853. Lapsed time—1-hour.

Bacterial Enumeration (CFU per mililitre)

| | Colony 1 | colony 2 | colony 3 | Average | 1-hrLog reduct'n | Reduction % |
|---|---|---|---|---|---|---|
| | 5,100,000 | 6,000,000 | 4,800,000 | 5,3000,000 | 6.7 | 99.99 |
| Control | 0 | 0 | 0 | 0 | | |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of the aforesaid steps or features.

1. A method for relieving pain or discomfort, and to promote healing of a subject wherein the pain or discomfort is caused by a medical condition or a physical injury to the soft tissues of the mouth and to the teeth, the method comprising the steps of: providing a composition consisting, by percentage weight, of: water—at least 70%; vegetable glycerine—about 10%; sea salt—about 12%; peppermint oil—at least 1.5%; and tea tree oil—at least 1.5%, and applying said composition to the affected soft tissue.

2. The method as set forth in claim 1, wherein said composition is applied by way of a mouth wash.

3. The method as set forth in claim 1, wherein said composition is applied by way of a spray.

4. The method as set forth in claim 1, wherein said composition is applied by painting directly onto said affected tissue by way of a swab.

5. A composition for use as a prophylaxis application in the treatment for periodontal infection: oral bone regeneration: apthous ulcers; herpes simplex and gingivitis, comprising, by percentage weight, of: water—at least 70%; vegetable glycerine—about 10%; sea salt—about 12%; peppermint oil—at least 1.5%; and tea tree oil—at least 1.5%.

6. The composition as set forth in claim 5, wherein said composition is a liquid solution.

7. The composition as set forth in claim 6, contained within an aerosol container.

8. The composition as set forth in claim 6, contained within a container having a pump.

9. The composition as set forth in claim 5, contained within a container having an applicator.

10. The composition as set forth in claim 9, wherein said container is a glass bottle.

11. The composition as set forth in claim 9, wherein said container is a coloured glass bottle.

12. The method as set forth in claim 1 wherein said composition consists, by percentage weight: water—about 70%; vegetable glycerine—about 10%; sea salt—about 12%; peppermint oil—about 2%; and tea tree oil—about 2%.

13. The method as set forth in claim 1 wherein said composition consists, by percentage weight: water—74.7%; vegetable glycerine—9.6%; sea salt—12.3%; peppermint oil—1.7%; and tea tree oil—1.7%.

14. The composition as set forth in claim 5, comprising, by percentage weight: water—74.7%; vegetable glycerine—9.6%; sea salt—12.3%; peppermint oil—1.7%; and tea tree oil—1.7%.
